(19) United States
(12) Patent Application Publication
(10) Pub. No.: US 2023/0043989 A1
(43) Pub. Date: Feb. 9, 2023

(54) Introducer Assemblies and Methods Thereof

(71) Applicant: Bard Access Systems, Inc., Salt Lake City, UT (US)

(72) Inventor: Glade H. Howell, Draper, UT (US)

(21) Appl. No.: 17/884,307

(22) Filed: Aug. 9, 2022

Related U.S. Application Data
(60) Provisional application No. 63/231,087, filed on Aug. 9, 2021.

(51) Int. Cl. A61M 25/09

(52) U.S. Cl. CPC A61M 25/09041

(57) ABSTRACT

Introducer assemblies and methods are disclosed. For example, an introducer assembly can include a syringe and a needle fluidly connected to the syringe. The syringe can include a barrel and a plunger disposed in the barrel. The plunger can include a one-piece plunger shaft and a piston fitted over a distal end of the plunger shaft. The plunger shaft can include a longitudinal plunger-shaft passageway extending from the distal end of the plunger shaft. The piston can include a piston through hole forming a portion of an access-guidewire passageway of the introducer assembly with the plunger-shaft passageway. The needle can include a needle shaft and a needle hub over a proximal portion of the needle shaft. In another example, a method is disclosed for securing vascular access with the foregoing introducer assembly.

### PRIORITY

This application claims the benefit of priority to U.S. Provisional Patent Application No. 63/231,087, filed Aug. 9, 2021, which is incorporated by reference in its entirety into this application.

### BACKGROUND

A guidewire is typically placed in a blood vessel with an introducer assembly before inserting a central venous catheter (“CVC”) or the like into the blood vessel over the guidewire. The introducer assembly typically includes a needle connected to a syringe. Upon accessing the blood vessel with the needle, the needle must be disconnected from the syringe to allow insertion of the guidewire into the needle through a needle hub thereof and, subsequently, into the blood vessel. Disconnecting the needle from the syringe as well as inserting the guidewire into the needle risk puncturing a backwall of the blood vessel, losing access to the blood vessel, or both due to overhandling the needle. What is needed is an introducer assembly that does not require disconnecting the needle from the syringe for inserting the guidewire into the blood vessel.

Disclosed herein are introducer assemblies and methods that address the foregoing.

### SUMMARY

Disclosed herein is an introducer assembly including, in some embodiments, a syringe and a needle fluidly connected to the syringe. The syringe includes a barrel and a plunger disposed in the barrel. The plunger includes a one-piece plunger shaft and a piston fitted over a distal end of the plunger shaft. The plunger shaft includes a longitudinal plunger-shaft passageway extending from the distal end of the plunger shaft. The piston includes a piston through hole forming a portion of an access-guidewire passageway of the introducer assembly with the plunger-shaft passageway. The needle includes a needle shaft and a needle hub over a proximal portion of the needle shaft.

In some embodiments, the introducer assembly further includes an access guidewire slidably disposed in the access-guidewire passageway. A needle lumen of the needle forms another portion of the access-guidewire passageway.

In some embodiments, the access guidewire includes a ‘J’-shaped guidewire tip in a distal portion of the access guidewire. A distal end of the access guidewire is disposed proximal of the needle tip in a ready-to-deploy state of the introducer assembly such that the guidewire tip assumes a straightened state in the ready-to-deploy state of the introducer assembly.

In some embodiments, the access guidewire includes a bare-wire portion and a wound-wire portion proximal of the bare-wire portion. The bare-wire portion distally extends from at least the piston through hole in a deployed state of the introducer assembly.

In some embodiments, the piston includes at least a leading ring. The leading ring includes a gasket around an inner perimeter of the piston through hole. The gasket is configured to form a seal around the access guidewire.

Also disclosed herein is an introducer assembly including, in some embodiments, a syringe and a needle fluidly connected to the syringe. The syringe includes a syringe hub, a barrel, and a plunger disposed in the barrel. The barrel includes a syringe tip extending from a distal portion of the barrel. The syringe tip includes a syringe-tip lumen extending from a distal end of the syringe tip into a barrel chamber of the barrel. The plunger includes a one-piece plunger shaft and a piston fitted over a distal end of the plunger shaft. The plunger shaft includes a longitudinal plunger-shaft passageway extending from the distal end of the plunger shaft. The piston includes a piston through hole. The needle includes a needle shaft and a needle hub over a proximal portion of the needle shaft. The needle shaft includes a needle tip in a distal portion of the needle shaft. The needle shaft also includes a needle-shaft lumen extending from an opening in the needle tip to a proximal end of the needle shaft. The needle hub includes a needle-hub lumen extending from a proximal end of the needle-shaft lumen to a proximal end of the needle hub. The needle-shaft lumen, the needle-hub lumen, the syringe-tip lumen, any intervening portion of the barrel chamber between the syringe tip and the piston, the piston through hole, and the plunger-shaft passageway are aligned to form an access-guidewire passageway. The access-guidewire passageway is configured to slidably accommodate an access guidewire therein.

In some embodiments, the introducer assembly further includes an access guidewire slidably disposed in the access-guidewire passageway. A distal end of the access guidewire is proximal of the needle tip in a ready-to-deploy state of the introducer assembly.

In some embodiments, the access guidewire includes a ‘J’-shaped guidewire tip in a distal portion of the access guidewire. The guidewire tip assumes a straightened state in the ready-to-deploy state of the introducer assembly. The guidewire tip assumes a curved state when the guidewire tip is advanced beyond the needle tip in a deployed state of the introducer assembly.

In some embodiments, the access guidewire includes a bare-wire portion and a wound-wire portion proximal of the bare-wire portion. The bare-wire portion distally extends from at least the piston through hole to the distal end of the access guidewire in the ready-to-deploy state of the introducer assembly.

In some embodiments, the piston includes at least a leading ring. The leading ring includes a gasket around an inner perimeter of the piston through hole. The gasket is configured to form a seal around the access guidewire.

In some embodiments, the gasket includes one or more integrated ‘0’-rings.

In some embodiments, the plunger shaft includes orthogonal struts meeting along their longitudinal edges at a central axis of the plunger shaft.

In some embodiments, the plunger-shaft passageway extends no more than about ½ a length of the plunger shaft from the distal end of the plunger shaft.

In some embodiments, the plunger-shaft passageway extends no more than about ⅓ a length of the plunger shaft from the distal end of the plunger shaft.

In some embodiments, the plunger-shaft passageway extends no more than about ¼ a length of the plunger shaft from the distal end of the plunger shaft.

In some embodiments, the syringe hub further includes a threaded collar extending from the distal portion of the barrel around the syringe tip. The threaded collar includes internal threads configured to screw together with a needle-hub flange of the needle hub.

Also disclosed herein is a syringe including, in some embodiments, a syringe hub, a barrel, and a plunger disposed in the barrel. The barrel includes a syringe tip extending from a distal portion of the barrel. The syringe tip includes a syringe-tip lumen extending from a distal end of the syringe tip into a barrel chamber of the barrel. The plunger includes a one-piece plunger shaft and a piston fitted over a distal end of the plunger shaft. The plunger shaft includes a longitudinal plunger-shaft passageway extending from the distal end of the plunger shaft. The piston includes a piston through hole. The syringe-tip lumen, any intervening portion of the barrel chamber between the syringe tip and the piston, the piston through hole, and the plunger-shaft passageway are aligned to form an access-guidewire passageway. The access-guidewire passageway is configured to slidably accommodate an access guidewire therein.

In some embodiments, the piston includes at least a leading ring. The leading ring includes a gasket around an inner perimeter of the piston through hole. The gasket is configured to form a seal around the access guidewire.

In some embodiments, the gasket includes one or more integrated ‘O’-rings.

In some embodiments, the plunger shaft includes orthogonal struts meeting along their longitudinal edges at a central axis of the plunger shaft.

In some embodiments, the plunger-shaft passageway extends no more than about ½ a length of the plunger shaft from the distal end of the plunger shaft.

In some embodiments, the plunger-shaft passageway extends no more than about ⅓ a length of the plunger shaft from the distal end of the plunger shaft.

In some embodiments, the plunger-shaft passageway extends no more than about ¼ a length of the plunger shaft from the distal end of the plunger shaft.

In some embodiments, the syringe hub further includes a threaded collar extending from the distal portion of the barrel around the syringe tip. The threaded collar includes internal threads configured to screw together with a needle-hub flange of a needle hub.

Also disclosed herein is a plunger for a syringe. The plunger includes, in some embodiments, a one-piece plunger shaft and a piston fitted over a distal end of the plunger shaft. The plunger shaft includes a longitudinal plunger-shaft passageway extending from the distal end of the plunger shaft. The piston includes a piston through hole. The piston through hole and the plunger-shaft passageway are aligned to form an access-guidewire passageway. The access-guidewire passageway is configured to slidably accommodate an access guidewire therein.

In some embodiments, the piston includes at least a leading ring. The leading ring includes a gasket around an inner perimeter of the piston through hole. The gasket is configured to form a seal around the access guidewire.

In some embodiments, the gasket includes one or more integrated ‘O’-rings.

In some embodiments, the plunger shaft includes orthogonal struts meeting along their longitudinal edges at a central axis of the plunger shaft.

In some embodiments, the plunger-shaft passageway extends no more than about ½ a length of the plunger shaft from the distal end of the plunger shaft.

In some embodiments, the plunger-shaft passageway extends no more than about ⅓ a length of the plunger shaft from the distal end of the plunger shaft.

In some embodiments, the plunger-shaft passageway extends no more than about ¼ a length of the plunger shaft from the distal end of the plunger shaft.

Also disclosed herein is a method for securing vascular access. The method includes, in some embodiments, an introducer assembly-obtaining step, a needle tract-establishing step, and an access guidewire-advancing step. The introducer assembly-obtaining step includes obtaining an introducer assembly. The introducer assembly includes a syringe, a needle fluidly connected to the syringe, and an access guidewire slidably disposed in an access-guidewire passageway of the introducer assembly. The syringe includes a barrel and a plunger disposed in the barrel. The needle includes a needle shaft and a needle hub over a proximal portion of the needle shaft. The access-guidewire passageway is formed from at least a needle lumen of the needle, a piston through hole of a piston fitted over a distal end of a plunger shaft of the plunger, and a plunger-shaft passageway extending from the distal end of the plunger shaft. The needle tract-establishing step includes establishing a needle tract from an area of skin to a blood-vessel lumen of a patient with the needle. The access guidewire-advancing step includes advancing the access guidewire into the blood-vessel lumen for the securing of the vascular access.

In some embodiments, the method further includes an introducer assembly-adjusting step. The introducer assembly-adjusting step includes adjusting the introducer assembly such that the introducer assembly is in a ready-to-deploy state thereof. In the ready-to-deploy state of the introducer assembly, a distal end of the access guidewire is disposed proximal of a needle tip of the needle for performing the access guidewire-advancing step immediately upon the establishing of the needle tract in the needle tract-establishing step.

In some embodiments, the access guidewire-advancing step allows a ‘J’-shaped guidewire tip in a distal portion of the access guidewire to transition from a straightened state in the access-guidewire passageway to a curved state in the blood-vessel lumen.

In some embodiments, the method further includes a blood-aspirating step. The blood-aspirating step includes aspirating blood with the syringe to confirm the establishing of the needle tract in the needle tract-establishing step. The piston through hole includes a gasket around an inner perimeter of the piston through hole. The gasket is configured to form a seal around a bare-wire portion of the access guidewire for maintaining a vacuum during the blood-aspirating step.

In some embodiments, the method further includes a needle-withdrawing step. The needle-withdrawing step includes withdrawing the needle from the patient leaving the access guidewire in the blood-vessel lumen.

These and other features of the concepts provided herein will become more apparent to those of skill in the art in view of the accompanying drawings and following description, which describe particular embodiments of such concepts in greater detail.

### DRAWINGS

FIG. 1 illustrates a side view of an introducer assembly in a ready-to-deploy state thereof in accordance with some embodiments.

FIG. 2 illustrates a side view of an introducer assembly in a deployed state thereof in accordance with some embodiments.

FIG. 3 illustrates a top view of the introducer assembly in the deployed state thereof in accordance with some embodiments.

FIG. 4 illustrates an exploded view of the introducer assembly in accordance with some embodiments.

FIG. 5 illustrates a side view of a plunger of a syringe of the introducer assembly in accordance with some embodiments.

FIG. 6 illustrates a longitudinal cross section of the plunger in accordance with some embodiments.

FIG. 7 illustrates a transverse cross section of a distal portion the plunger in accordance with some embodiments.

FIG. 8 illustrates a transverse cross section of a proximal portion of the plunger in accordance with some embodiments.

FIG. 9 illustrates a detailed view of the distal portion of the plunger including a longitudinal plunger-shaft passageway of a plunger shaft of the plunger and a piston through hole of a piston of the plunger in accordance with some embodiments.

FIG. 10 illustrates a detailed view of the distal portion of the plunger including an access guidewire disposed in the longitudinal plunger-shaft passageway and the piston through hole in accordance with some embodiments.

FIG. 11 illustrates a detailed view of a longitudinal cross section of the introducer assembly with the access guidewire disposed in an access-guidewire passageway of the introducer assembly in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, a guidewire is typically placed in a blood vessel with an introducer assembly before inserting a central venous catheter (“CVC”) or the like into the blood vessel over the guidewire. The introducer assembly typically includes a needle connected to a syringe. Upon accessing the blood vessel with the needle, the needle must be disconnected from the syringe to allow insertion of the guidewire into the needle through a needle hub thereof and, subsequently, into the blood vessel. Disconnecting the needle from the syringe as well as inserting the guidewire into the needle risk puncturing a backwall of the blood vessel, losing access to the blood vessel, or both due to overhandling the needle. What is needed is an introducer assembly that does not require disconnecting the needle from the syringe for inserting the guidewire into the blood vessel.

Disclosed herein are introducer assemblies and methods that do not require disconnecting the needle from the syringe for inserting the guidewire into a blood vessel like typical introducer assemblies. Such introducer assemblies and methods are advantageous in that they do not have the same risk of puncturing a backwall of the blood vessel or losing access to the blood vessel due to overhandling. In an example, an introducer assembly is disclosed including, in some embodiments, a syringe and a needle fluidly connected to the syringe. The syringe includes a barrel and a plunger disposed in the barrel. The plunger includes a one-piece plunger shaft and a piston fitted over a distal end of the plunger shaft. The plunger shaft includes a longitudinal plunger-shaft passageway extending from the distal end of the plunger shaft. The piston includes a piston through hole forming a portion of an access-guidewire passageway of the introducer assembly with the plunger-shaft passageway. The needle includes a needle shaft and a needle hub over a proximal portion of the needle shaft. In another example, a method is disclosed for securing vascular access with the foregoing introducer assembly. Again, these and other features will become more apparent in view of the accompanying drawings and following description, which describe particular embodiments in greater detail.

### Introducer Assemblies

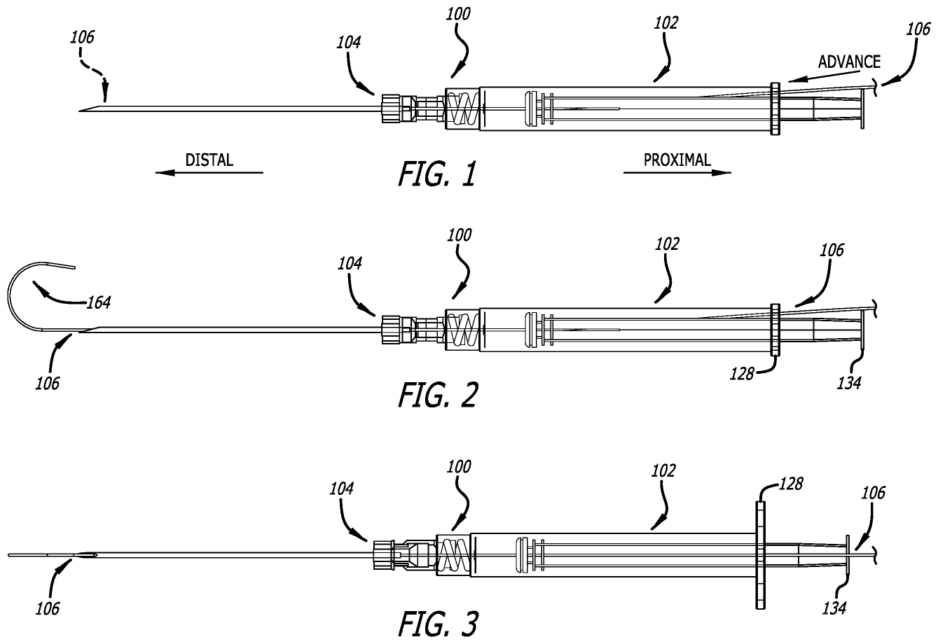

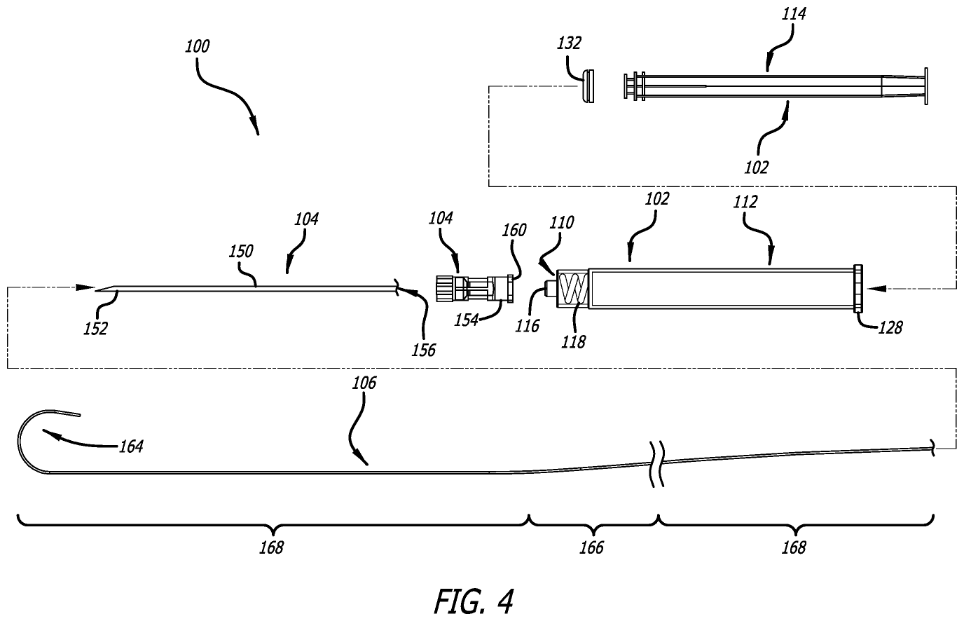

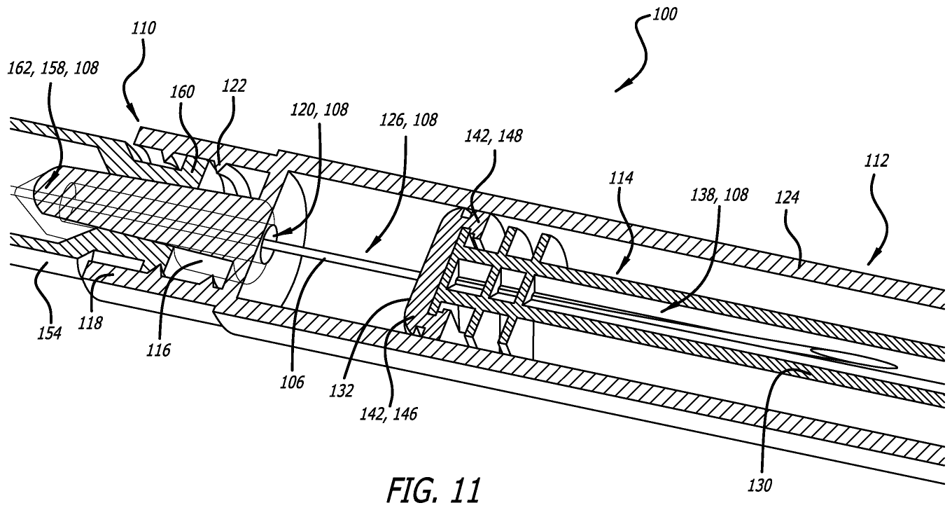

FIGS. 1-4 and 11 illustrate various views of an introducer assembly 100 in accordance with some embodiments.

As shown, the introducer assembly 100 includes at least a syringe 102 and a needle 104 fluidly connected to the syringe 102. In addition, the introducer assembly 100 can include an access guidewire 106 slidably disposed in an access-guidewire passageway 108 of the introducer assembly 100. Such an introducer assembly with the access guidewire 106 preloaded in the access-guidewire passageway 108 is advantageous with respect to obviating any difficulties with loading the access guidewire 106 into the introducer assembly 100 as well as lessening procedure time by an amount commensurate with the loading of the access guidewire 106 into the introducer assembly 100. Notwithstanding the foregoing advantages, the introducer assembly 100 need not include the access guidewire 106 preloaded in the access-guidewire passageway 108 in some embodiments.

The syringe 102 includes a syringe hub 110, a barrel 112, and a plunger 114 disposed in the barrel 112.

The syringe hub 110 includes a syringe tip 116 extending from a distal portion (e.g., a distal end) of the barrel 112. In addition, the syringe hub 110 can include a threaded collar 118 extending from the distal portion (e.g., the distal end) of the barrel 112 around the syringe tip 116.

The syringe tip 116 is configured to insert into the needle hub 154 set forth below for fluidly connecting the syringe 102 and the needle 104. Indeed, the syringe tip 116 can have a Luer taper (e.g., a 6% taper) configured to insert into the needle hub 154, which needle hub 154 is complementarily configured as set forth below.

The syringe tip 116 includes a syringe-tip lumen 120 extending from a distal end of the syringe tip 116 into the barrel chamber 126 of the barrel 112 set forth below. Notably, the syringe-tip lumen 120 and the barrel chamber 126 independently and collectively form a portion of the access-guidewire passageway 108 of the introducer assembly 100. When considered collectively, the syringe-tip lumen 120 and the barrel chamber 126 form a hub-and-barrel portion of the access-guidewire passageway 108 of the introducer assembly 100.

The threaded collar 118 includes internal threads 122 configured to screw together with the needle-hub flange 160 of the needle hub 154 set forth below. When present, the threaded collar 118 of the syringe hub 110 advantageously provides a so-called Luer lock-style connection with the needle-hub flange 160 of the needle hub 154 for added security against inadvertent disconnection over that provided by an otherwise Luer slip-style connection between the syringe hub 110 and the needle hub 154.

The barrel 112 includes a barrel wall 124, a barrel chamber 126 defined by the barrel wall 124, and a barrel flange 128, barrel collar, or the like outwardly extending from a proximal portion (e.g., a proximal end) of the barrel 112 or barrel wall 124 configured for actuating the syringe 102.

The barrel chamber 126 is configured to accept the plunger 114 when inserted therein. Indeed, the barrel chamber 126 extends from the distal end of the barrel 112, which is a closed end of the barrel 112 (excepting the syringe tip 116), to the proximal end of the barrel 112, which is an open end of the barrel 112 into which the plunger 114 can be inserted. Again, the syringe-tip lumen 120 and the barrel chamber 126—particularly any intervening portion of the barrel chamber 126 between the syringe tip 116 and the piston 132 set forth below—independently and collectively form a portion of the access-guidewire passageway 108 of the introducer assembly 100, particularly the hub-and-barrel portion of the access-guidewire passageway 108. That said, any remaining portion of the barrel chamber 126 between the piston 132 and the barrel flange 128, barrel collar, or the like can also be considered a portion of the access-guidewire passageway 108 of the introducer assembly 100, albeit a less constrained portion of the access-guidewire passageway 108 as shown in FIGS. 1-4 and 11.

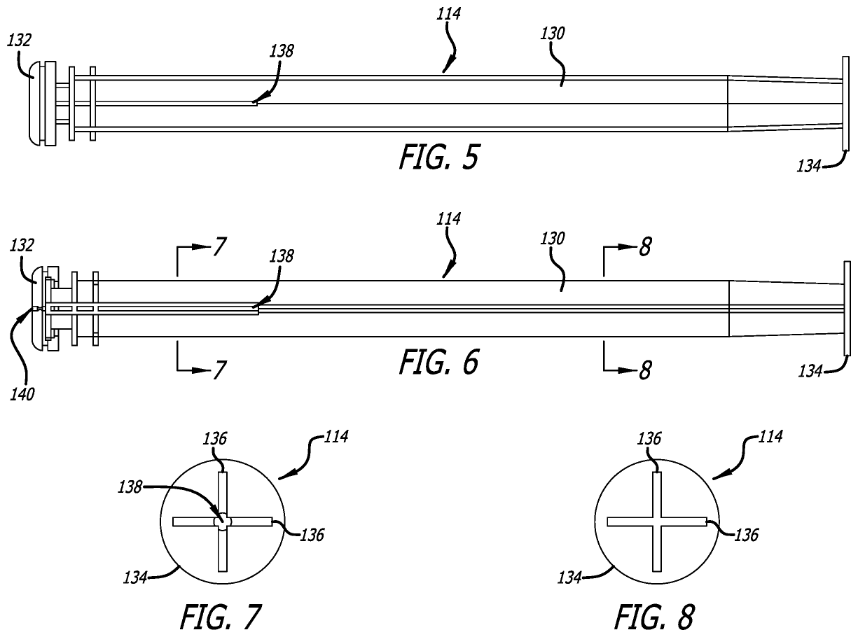

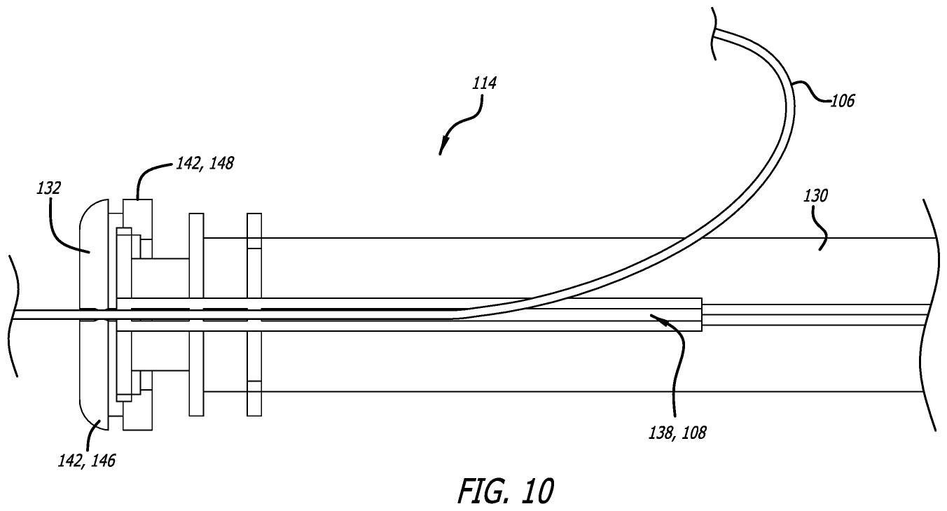

FIGS. 5-10 illustrate various views of the plunger 114 of the syringe 102 of the introducer assembly 100 in accordance with some embodiments.

As shown, the plunger 114 includes a one-piece plunger shaft 130, a piston 132 fitted over a distal portion (e.g., a distal end) of the plunger shaft 130, and a plunger flange 134, a plunger collar, or the like outwardly extending from a proximal portion (e.g., a proximal end) of the plunger 114 configured for actuating the syringe 102.

The plunger shaft 130 includes orthogonal struts 136 meeting along their longitudinal edges at a central axis of the plunger shaft 130 except for in the distal portion of the plunger shaft 130, which includes a longitudinal plunger-shaft passageway 138 extending from the distal end of the plunger shaft 130. The plunger-shaft passageway 138 extends no more than about ½ a length of the plunger shaft 130 from the distal end of the plunger shaft 130, including no more than about ⅓ a length of the plunger shaft 130 from the distal end of the plunger shaft 130, such as no more than about ¼ a length of the plunger shaft 130 from the distal end of the plunger shaft 130. Notably, the plunger-shaft passageway 138 is configured to form a portion of the access-guidewire passageway 108 of the introducer assembly 100 independently and collectively with the piston through hole 140 set forth below. When considered collectively, the plunger-shaft passageway 138 and the piston through hole 140 form a plunger portion of the access-guidewire passageway 108 of the introducer assembly 100.

While not shown, the plunger shaft 130 can alternatively be configured as an open cylinder like the barrel 112 or a closed cylinder like a rod, wherein the proximal end of such a plunger shaft is respectively open or closed. When the alternative plunger shaft is configured as the open cylinder, the distal end of the plunger shaft, which is otherwise closed, includes a plunger-shaft through hole configured to pass the access guidewire 106 through the plunger-shaft through hole, into an internal cavity or chamber of the plunger shaft defined by a plunger-shaft wall, and out the proximal end of the alternative plunger shaft. When the alternative plunger shaft is configured as the closed cylinder, the alternative plunger shaft can be either hollow or solid. For the alternative plunger shaft configured as the hollow closed cylinder, the distal end of the alternative plunger shaft, which is otherwise closed, includes a distal plunger-shaft through hole. Likewise, the proximal end of the alternative plunger shaft, which is otherwise closed, includes a proximal plunger-shaft through hole. Such an alternative plunger shaft is configured to pass the access guidewire 106 through the distal plunger-shaft through hole, into the internal cavity or chamber of the alternative plunger shaft defined by the plunger-shaft wall, and out the distal plunger-shaft through hole. For the alternative plunger shaft configured as the solid closed cylinder, the alternative plunger shaft includes both of the foregoing distal and proximal plunger-shaft through holes; however, the distal and proximal plunger-shaft through holes of the alternative plunger shaft configured as the solid closed cylinder are connected by a plunger-shaft lumen through the alternative plunger shaft—not the internal chamber or cavity of the alternative plunger shaft configured as the hollow closed cylinder. Regardless, as set forth above, the alternative plunger shaft remains a one-piece plunger shaft with a plunger-shaft passageway.

The piston 132, which can be an integral, elastomeric piston, includes a piston through hole 140, one or more rings 142 configured to respectively form one or more seals with the barrel wall 124, and one or more gaskets 144 configured to respectively form one or more seals around the access guidewire 106.

The piston through hole 140 is configured to form a portion of the access-guidewire passageway 108 of the introducer assembly 100 independently and collectively with the plunger-shaft passageway 138. When considered collectively, the piston through hole 140 and the plunger-shaft passageway 138 form the plunger portion of the access-guidewire passageway 108 of the introducer assembly 100. Notably, the plunger and hub-and-barrel portions of the access-guidewire passageway 108 collectively form a syringe portion of the access-guidewire passageway 108 of the introducer assembly 100. The syringe portion of the access-guidewire passageway 108, in turn, collectively forms the access-guidewire passageway 108 of the introducer assembly 100 with the needle portion of the access-guidewire passageway 108 set forth below.

The one-or-more rings 142 include at least a leading ring 146 configured to form a seal with the barrel wall 124. The one-or-more rings 142 can also include a trailing ring 148 as shown in FIGS. 5-10. Like the leading ring 146, the trailing ring 148 is configured to form a seal with the barrel wall 124. Indeed, the trailing ring 148, when present, provides a backup seal with the barrel wall 124. Together, the leading ring 146 and the trailing ring 148 ensure the seal (e.g., the seal provided by the leading ring 146, the trailing ring 148, or both the leading ring 146 and the trailing ring 148) between the piston 132 and the barrel wall 124 remains intact while the syringe 102 is actuated, thereby allowing the syringe 102 to consistently aspirate a liquid such as blood when the plunger 114 is withdrawn from the barrel 112.

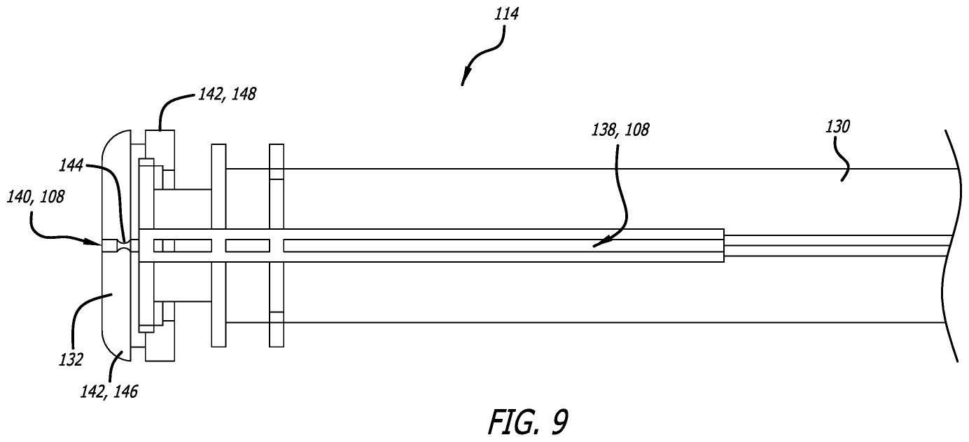

The one-or-more gaskets 144 can be disposed in the piston through hole 140, over a distal end of the piston 132 around the piston through hole 140, over a medial portion of the piston 132 around the piston through hole 140, or a combination thereof for forming a seal around the access guidewire 106 (e.g., the bare-wire portion 166 of the access guidewire 106 set forth below). The one-or-more gaskets 144 can include one or more integrated ‘O’-rings, wherein the one-or-more integrated ‘O’-rings are molded into the piston 132 at a time the piston 132 is molded. In the example shown in FIG. 9, a single gasket is configured as an integrated ‘O’-ring disposed in the piston through hole 140. Indeed, the leading ring 146 of the piston 132 includes the gasket around an inner perimeter of the piston through hole 140 for forming a seal around the access guidewire 106.

FIGS. 1-4 illustrate various views of the needle 104 as part of the introducer assembly 100 in accordance with some embodiments.

As shown, the needle 104 includes a needle shaft 150, a needle tip 152 in a distal portion of the needle shaft 150, and a needle hub 154 over a proximal portion of the needle shaft 150.

The needle shaft 150 includes a needle-shaft lumen 156 extending from an opening in the needle tip 152 to a proximal end of the needle shaft 150. Notably, the needle-shaft lumen 156 and the needle-hub lumen 162 set forth below independently and collectively form a portion of the access-guidewire passageway 108 of the introducer assembly 100. When considered collectively, the needle-shaft lumen 156 and the needle-hub lumen 162 form a needle portion of the access-guidewire passageway 108 of the introducer assembly 100.

The needle hub 154 is configured to accept the syringe tip 116 therein for fluidly connecting the needle 104 and the syringe 102. Indeed, a bore 158 of the needle hub 154 can have a Luer taper (e.g., a 6% taper) configured to accept the syringe tip 116 therein, which syringe tip 116 is complementarily configured as set forth above. In addition, the needle hub 154 can include a needle-hub flange 160 configured to screw together with the internal threads 122 of the threaded collar 118 of the syringe hub 110. When present, the needle-hub flange 160 of the needle hub 154 advantageously provides a so-called Luer lock-style connection with the internal threads 122 of the threaded collar 118 of the syringe hub 110 for added security against inadvertent disconnection over that provided by an otherwise Luer slip-style connection between the needle hub 154 and the syringe hub 110.

The needle hub 154 includes a needle-hub lumen 162 extending from a distal end of the needle hub 154 to a proximal end of the needle hub 154; however, with the needle hub 154 over the proximal portion of the needle shaft 150, the needle-hub lumen 162 practically extends from a proximal end of the needle-shaft lumen 156 to the proximal end of the needle hub 154. Again, the needle-shaft lumen 156 and the needle-hub lumen 162 independently and collectively form a portion of the access-guidewire passageway 108 of the introducer assembly 100, particularly the needle portion of the access-guidewire passageway 108.

In summary of description set forth above for the access-guidewire passageway 108, the access-guidewire passageway 108 of the introducer assembly 100 includes both the syringe and needle portions of the access-guidewire passageway 108 when the syringe 102 and needle 104 are fluidly connected together. The syringe portion of the access-guidewire passageway 108 includes both the hub-and-barrel and plunger portions of the access-guidewire passageway 108 when the plunger 114 is disposed in the barrel 112, wherein the syringe-tip lumen 120 and the barrel chamber 126 form the hub-and-barrel portion of the access-guidewire passageway 108, and wherein the piston through hole 140 and the plunger-shaft passageway 138 form the plunger portion of the access-guidewire passageway 108. The needle portion of the access-guidewire passageway 108 includes both the needle-shaft lumen 156 and the needle-hub lumen 162. As such, the needle-shaft lumen 156, the needle-hub lumen 162, the syringe-tip lumen 120, any intervening portion of the barrel chamber 126 between the syringe tip 116 and the piston 132, the piston through hole 140, and the plunger-shaft passageway 138 when aligned, for example, in a fluidly connected, ready-to-deploy state of the introducer assembly 100, form the access-guidewire passageway 108 of the introducer assembly 100. The access-guidewire passageway 108 is configured to slidably accommodate the access guidewire 106 therein.

FIGS. 1-4 illustrate various views of the access guidewire 106 as part of the introducer assembly 100 in accordance with some embodiments.

As shown, the access guidewire 106 is slidably disposed in the access-guidewire passageway 108 of the introducer assembly 100 in an assembled state of the introducer assembly 100 such as the ready-to-deploy state of the introducer assembly 100. Indeed, in the ready-to-deploy state of the introducer assembly 100 shown in FIG. 1, a distal end of the access guidewire 106 is just proximal of the needle tip 152 such that the access guidewire 106 can be immediately advanced a centimeter or less into a blood-vessel lumen to secure vascular access upon establishing a needle tract to the blood vessel. A remainder of the access guidewire 106 in the ready-to-deploy state of the introducer assembly 100 proximally extends along the access-guidewire passageway 108 and exits past the barrel flange 128, barrel collar, or the like.

The access guidewire 106 can include a ‘J’-shaped guidewire tip 164 in a distal portion of the access guidewire 106. The guidewire tip 164 is configured to assume a straightened state in the ready-to-deploy state of the introducer assembly 100, in which, again, the distal end of the access guidewire 106 is just proximal of the needle tip 152. However, the guidewire tip 164 is configured to assume a curved state or ‘J’ shape when the guidewire tip 164 is advanced beyond the needle tip 152 in a deployed state of the introducer assembly 100 such as when the access guidewire 106 is advanced into a blood-vessel lumen to secure vascular access upon establishing a needle tract to the blood vessel.

The access guidewire 106 can also include a bare-wire portion 166 and a wound-wire portion 168 distal of the bare-wire portion 166, proximal of the bare-wire portion 166, or both. When present, the bare-wire portion 166 distally extends from at least proximal of the piston through hole 140 to a more distal portion of the access guidewire 106 in the ready-to-deploy state of the introducer assembly 100. Indeed, the bare-wire portion 166 also distally extends from at least proximal of the piston through hole 140 to the more distal portion of the access guidewire 106 in the foregoing deployed state of the introducer assembly 100, in which the access guidewire 106 is advanced into the blood-vessel lumen. Such a bare-wire portion of the access guidewire 106 allows the one-or-more gaskets 144 of the piston 132 to consistently form the one-or-more seals around the access guidewire 106. That said, the wound-wire portion 168, which provides structural integrity to the access guidewire 106, can extend through the piston through hole 140 in one or more operable states of the introducer assembly 100 or even over an entirety of the access guidewire 106 in some embodiments. In such embodiments, it is advantageous for windings of the wound-wire portion 168 of the access guidewire 106 to be as tight as possible to minimize any air-leaking gaps between adjacent windings that might frustrate forming the one-or-more seals around the access guidewire 106 with the one-or-more gaskets 144. While the air-leaking gaps for any portion of the wound-wire portion 168 of the access guidewire 106 configured to extend through the piston through hole 140 can be minimized using a small-diameter winding wire around a core wire of the access guidewire 106, the foregoing wound-wire portion 168 of the access guidewire 106 can alternatively be a flat-wound or ground-wound portion of the access guidewire 106.

While not shown, the access guidewire 106 exiting past the barrel flange 128, barrel collar, or the like in the ready-to-deploy state of the introducer assembly 100 can be disposed in a sterile barrier such as sterile bag to maintain sterility of the access guidewire 106 prior to deploying the access guidewire 106.

### Methods

Methods of the introducer assembly 100 include methods of using the introducer assembly 100 to secure vascular access. For example, a method to secure vascular access includes one or more step selected from an introducer assembly-obtaining step, an introducer assembly-adjusting step, a needle tract-establishing step, a blood-aspirating step, an access guidewire-advancing step, and a needle-withdrawing step.

The introducer assembly-obtaining step includes obtaining the introducer assembly 100. As set forth above, the introducer assembly 100 includes the syringe 102, the needle 104 fluidly connected to the syringe 102, and the access guidewire 106 slidably disposed in the access-guidewire passageway 108 of the introducer assembly 100 in at least the ready-to-deploy state of the introducer assembly 100. The syringe 102 includes the barrel 112 and the plunger 114 disposed in the barrel 112. The needle 104 includes the needle shaft 150 and the needle hub 154 over the proximal portion of the needle shaft 150. The access-guidewire passageway 108 is formed from at least the needle lumen of the needle 104, the piston through hole 140 of the piston 132 fitted over the distal end of the plunger shaft 130 of the plunger 114, and the plunger-shaft passageway 138 extending from the distal end of the plunger shaft 130.

The introducer assembly-adjusting step includes adjusting the introducer assembly 100 such that the introducer assembly 100 is in the ready-to-deploy state thereof if not already in the ready-to-deploy state. In the ready-to-deploy state of the introducer assembly 100, the distal end of the access guidewire 106 is just proximal of the needle tip 152 of the needle 104 for performing the access guidewire-advancing step immediately upon the establishing of the needle tract in the needle tract-establishing step.

The needle tract-establishing step includes establishing a needle tract from an area of skin to a blood-vessel lumen of a patient with the needle 104. Notably, blood flashback is often witnessed upon establishing the needle tract due to an annular space between an inner diameter of the needle shaft 150 and an outer diameter of the access guidewire 106, particularly the bare-wire portion 166 of the access guidewire 106 or the corresponding wire-wound portion of the access guidewire 106 configured with minimal to no air-leaking gaps between the adjacent windings.

The blood-aspirating step includes aspirating blood with the syringe 102 to confirm the establishing of the needle tract in the needle tract-establishing step. As set forth above, the piston through hole 140 can include the gasket around the inner perimeter of the piston through hole 140. The gasket is configured to form a seal around at least the bare-wire portion 166 of the access guidewire 106 for maintaining a vacuum during the blood-aspirating step. Notably, blood is drawn with the access guidewire 106 in the introducer assembly 100.

The access guidewire-advancing step includes advancing the access guidewire 106 into the blood-vessel lumen for the securing of the vascular access. The access guidewire-advancing step allows the ‘J’-shaped guidewire tip 164 in the distal portion of the access guidewire 106 to transition from the straightened state in the access-guidewire passageway 108 to the curved state in the blood-vessel lumen.

The needle-withdrawing step includes withdrawing the needle 104 from the patient leaving the access guidewire 106 in the blood-vessel lumen.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An introducer assembly, comprising:
a syringe, including:
a barrel; and
a plunger disposed in the barrel, the plunger including:
a one-piece plunger shaft including a longitudinal plunger-shaft passageway extending from a distal end of the plunger shaft; and
a piston fitted over the distal end of the plunger shaft, the piston including a piston through hole forming a portion of an access-guidewire passageway of the introducer assembly with the plunger-shaft passageway;
a needle fluidly connected to the syringe, the needle including:
a needle shaft; and
a needle hub over a proximal portion of the needle shaft.

2. The introducer assembly of claim 1, further comprising an access guidewire slidably disposed in the access-guidewire passageway, a needle lumen of the needle forming another portion of the access-guidewire passageway.

3. The introducer assembly of claim 2, wherein the access guidewire includes a ‘J’-shaped guidewire tip in a distal portion of the access guidewire, a distal end of the access guidewire disposed proximal of the needle tip in a ready-to-deploy state of the introducer assembly such that the guidewire tip assumes a straightened state in the ready-to-deploy state of the introducer assembly.

4. The introducer assembly of claim 2, wherein the access guidewire includes a bare-wire portion and a wound-wire portion proximal of the bare-wire portion, the bare-wire portion distally extending from at least the piston through hole in a deployed state of the introducer assembly.

5. The introducer assembly of claim 1, wherein the piston includes at least a leading ring, the leading ring including a gasket around an inner perimeter of the piston through hole configured to form a seal around the access guidewire.

6. An introducer assembly, comprising:
a syringe, including:
a barrel;
a syringe hub including a syringe tip extending from a distal portion of the barrel, the syringe tip including a syringe-tip lumen extending from a distal end of the syringe tip into a barrel chamber of the barrel; and
a plunger disposed in the barrel, the plunger including:
a one-piece plunger shaft including a longitudinal plunger-shaft passageway extending from a distal end of the plunger shaft; and
a piston fitted over the distal end of the plunger shaft, the piston including a piston through hole; and
a needle fluidly connected to the syringe, the needle including:
a needle shaft including a needle tip in a distal portion of the needle shaft, the needle shaft including a needle-shaft lumen extending from an opening in the needle tip to a proximal end of the needle shaft; and
a needle hub over a proximal portion of the needle shaft, the needle hub including a needle-hub lumen extending from a proximal end of the needle-shaft lumen to a proximal end of the needle hub, the needle-shaft lumen, the needle-hub lumen, the syringe-tip lumen, any intervening portion of the barrel chamber between the syringe tip and the piston, the piston through hole, and the plunger-shaft passageway aligned to form an access-guidewire passageway configured to slidably accommodate an access guidewire therein.

7. The introducer assembly of claim 6, further comprising an access guidewire slidably disposed in the access-guidewire passageway, a distal end of the access guidewire proximal of the needle tip in a ready-to-deploy state of the introducer assembly.

8. The introducer assembly of claim 7, wherein the access guidewire includes a ‘J’-shaped guidewire tip in a distal portion of the access guidewire, the guidewire tip assuming a straightened state in the ready-to-deploy state of the introducer assembly and a curved state when the guidewire tip is advanced beyond the needle tip in a deployed state of the introducer assembly.

9. The introducer assembly of claim 7, wherein the access guidewire includes a bare-wire portion and a wound-wire portion proximal of the bare-wire portion, the bare-wire portion extending from at least the piston through hole to the distal end of the access guidewire in the ready-to-deploy state of the introducer assembly.

10. The introducer assembly of claim 6, wherein the piston includes at least a leading ring, the leading ring including a gasket around an inner perimeter of the piston through hole configured to form a seal around the access guidewire.

11. The introducer assembly of claim 10, wherein the gasket includes one or more integrated ‘O’-rings.

12. The introducer assembly of claim 6, wherein the plunger shaft includes orthogonal struts meeting along their longitudinal edges at a central axis of the plunger shaft.

13. The introducer assembly of claim 6, wherein the plunger-shaft passageway extends no more than about ½ a length of the plunger shaft from the distal end of the plunger shaft.

14. The introducer assembly of claim 6, wherein the plunger-shaft passageway extends no more than about ⅓ a length of the plunger shaft from the distal end of the plunger shaft.

15. The introducer assembly of claim 6, wherein the plunger-shaft passageway extends no more than about ¼ a length of the plunger shaft from the distal end of the plunger shaft.

16. The introducer assembly of claim 6, the syringe hub further including a threaded collar extending from the distal portion of the barrel around the syringe tip, the threaded collar including internal threads configured to screw together with a needle-hub flange of the needle hub.

17-36. (canceled)
